(19) United States
(12) Patent Application Publication
(10) Pub. No.: US 2026/0234708 A1
(43) Pub. Date: Aug. 13, 2026

(54) NUCLEIC ACID AMPLIFICATION CASSETTE

(71) Applicants: Testmate Health SA, Vaud (CH); CSEM CENTRE SUISSE D'ELECTRONIQUE ET DE MICROTECHNIQUE SA, Neuchâtel (CH)

(72) Inventors: Hüseyin Baris Atakan, Rotkreuz (CH); Noa Schmid, Alpnach (CH); Roger Limacher, Vaud (CH); Georg Orawez, Vaud (CH); Stephan Bitterli, Vaud (CH); Elie Graham, Rolle (CH); Gaëtan Cherbuin, Chavannes-près-Renens (CH)

(21) Appl. No.: 19/152,915

(22) PCT Filed: Jan. 31, 2024
(86) PCT No.: PCT/EP2024/052324

§ 371 Date: Jul. 31, 2025

(30) Foreign Application Priority Data
Jan. 31, 2023 (EP) 23154288.7

(51) Int. Cl. C12Q 1/6844; B01L 3/00; B01L 7/00; C12Q 1/689; C12Q 1/6893

(52) U.S. Cl. CPC C12Q 1/6844; B01L 3/502715; B01L 7/00; C12Q 1/689; C12Q 1/6893; B01L 2200/027; B01L 2200/16; B01L 2300/0627; B01L 2300/0825; B01L 2300/0864; C12Q 2600/112

(57) ABSTRACT

The present disclosure relates to a system and method for a nucleic acid amplification device for testing a bodily fluid sample for illness. In some embodiments, the device comprises a housing having an inlet port and an incubation and detection section configured to enable the amplification and the detection of nucleic acids. At least one first microfluidic conduit connects the inlet port with at least one incubation chamber. At least one nucleic acid amplification composition including a primer configured to amplify nucleic acids indicative of an illness, is located along the first microfluidic conduit and/or in the incubation chamber. In some embodiments, the device further comprises a heating arrangement comprising a heating element adjacent the at least one incubation chamber.

### CROSS REFERENCE TO RELATED APPLICATIONS

This application claims priority to and the benefit of European Application No. EP 23 154 288.7, filed on Jan. 31, 2023, the entire contents of which are incorporated by reference herein for all purposes.

### BACKGROUND

The present disclosure relates to a nucleic acid amplification device for testing a bodily fluid sample for illness.

Sexually transmitted infections (STIs) represent several of the most frequently reported diseases in Europe and in the United States and, notably, the incidence of chlamydia and gonorrhea have increased in recent years. Notably, during the Covid-19 pandemic access to clinics and doctors was sharpy curtailed. Furthermore, many individuals were hesitant to have in-person appointments with healthcare providers given the risk of Covid-19 transmission. As such, experts believe that many sexually transmitted infections went unidentified during this time. Many patients with a sexually transmitted infection can be asymptomatic and further transmit the infection before the infection is diagnosed and treated. For some the stigma associated with STIs can present barriers to patients seeking treatment. Even those who seek testing and treatment may have difficulty with access to testing and delays in receiving test results. Altogether these factors provide numerous obstacles to diagnosis and treatment which put individuals at risk for acquiring a sexually transmitted infection or having further complications from unidentified infections.

The testing market is changing to address some of these barriers by increasing the speed of testing, such a same-day testing being available in doctor's offices and clinics. Existing diagnostic kits designed for STD testing in clinical settings, however, rely on numerous reusable instruments and temperature sensitive reagents that are unsuitable for retail sale and home use.

Presently, some at home STI testing kits exist. However, these kits require the user to take a sample and then mail the sample to a central lab for processing. These home sampling kits generally take days or weeks to receive the results. Thus, a need exists for a STI testing device which can be performed in the privacy of a person's home and also provide fast at-home results.

Consequently, it is an object of the present disclosure to provide a system which provides an improvement over known processes and enables the amplification and identification of DNA or RNA amplification from bodily fluids in order to detect illness, in particular a system suitable for retail sale and/or home use. It is also an object of the present disclosure to provide a device which provides an improvement over known processes and addresses the problems described.

### SUMMARY

The present disclosure is achieved, inter alia, with the features of the independent claims. Dependent claims refer to preferred embodiments.

According to a first aspect, the present disclosure relates to nucleic acid amplification device for testing a bodily fluid sample for illness. The device comprises a housing comprising an inlet port for receiving a sample derived from a bodily fluid, an incubation and detection section configured to enable the amplification and the detection of nucleic acids, the section comprising at least one incubation chamber. The device also comprises one or more first microfluidic conduits connecting the inlet port with each of the one or more incubation chambers and at least one nucleic acid amplification composition including a primer configured to amplify nucleic acids indicative of an illness, each of the nucleic acid amplification compositions being located along each of the first microfluidic conduits and/or in each incubation chamber.

Preferably, the incubation and detection section comprises a plurality of incubation chambers, wherein the device comprises a plurality of first microfluidic conduits connecting the inlet port with each of the incubation chambers and a plurality of nucleic acid amplification compositions, each including a primer configured to amplify nucleic acids indicative of an illness, each of the nucleic acid amplification compositions being located along each of the first microfluidic conduits and/or in each incubation chamber.

Preferably, the nucleic acid amplification compositions are provided in the form of a lyophilized pellet, sphere or powder, i.e. as a plurality of lyophilized pellets, spheres or powders. As noted above, existing diagnostic kits designed for STD testing in clinical settings frequently rely on various temperature sensitive reagents. Such reagents often require storage at different temperatures, usually 4° C. and −20° C., and sometimes −80° C. This makes the devices unsuitable for retail sale and/or home use. By using a plurality of lyophilized pellets, spheres or powders, the present disclosure provides a nucleic acid amplification device including reagents which are stable at room temperature, e.g. for over a year. This makes the amplification device suitable for retail sale and home use as well as being suitable for use in low resource environments.

The amplification device as described herein enables testing for one or more illnesses. An illness as described herein may be understood to be any type of infection or condition which can be identified by the presence of DNA or RNA within a bodily fluid. Specific implementations of the present disclosure are for the detection of sexually transmitted diseases, such as infections caused by bacteria (e.g. chlamydia or gonorrhea), infections caused by protists (e.g. trichomonas vaginalis), oncological conditions (e.g. cancerous or precancerous conditions which can be determined based on the presence of cancerous or precancerous cells in the urine, saliva or mouthwash), bacterial infections (such as urinary tract infection, urethritis, cystitis, pyelonephritis, gingivitis, and/or periodontitis), fungal infections, and/or protist infections.

Preferably, the incubation and detection section further comprises a plurality of detection chambers configured to enable the detection of amplified nucleic acids, and the incubation and detection section further comprises a plurality of second microfluidic conduits connecting each of the incubation chambers with a respective detection chamber. Alternatively, detection could occur via the one or more incubations chambers in some cases.

Preferably, the device comprises at least 2, at least 3 or at least 5 incubation chambers. Alternatively, the device comprises between 2 and 12 incubation chambers, preferably between 2 and 7, more preferably between 3 and 7, most preferably 5 incubation chambers. Providing a plurality of parallel incubation tracks for the sample enables simultaneous amplification of a number of different DNA or RNA segments. This enables the user to test for a variety of different illnesses using a single sample (i.e., multiplexing).

Preferably, each nucleic acid amplification composition comprises a different primer configured to amplify nucleic acids indicative of a different illness. The primer may be configured to amplify nucleic acids of at least one of Chlamydia trachomatis, Neisseria gonorrhoeae, Mycoplasma genitalium or Trichomonas vaginalis. As some of the most common sexually transmitted infections, providing primers for each in separate incubation chambers allows the user to test for multiple STIs using one sample.

Preferably, the incubation and detection section comprises at least one test composition. The test composition may be provided in a test strip, as a solid test material (e.g., a test powder), or as a test liquid. The test composition preferably is functionalized to change in color upon presence of a threshold amount of DNA and/or RNA. In comparison to prior DNA/RNA detection methods, providing a color change detection method enables read-out of results by a layperson without specialized lab equipment. The test composition may be provided in the one or more incubation chambers and/or in the one or more detection chambers. For example, a test strip (e.g., with a functionalized or adsorbed leuco dye) may be provided in each of the detection chambers or a solid test material (such as a powder) may be provided inside and/or adhered to the walls of the various incubation chambers and/or detection chambers.

In some embodiments, the test composition (e.g., the test strip, solid test material, or test liquid) comprises a leuco dye, preferably a leuco dye configured to change in color (e.g., to turn to a blue color) to enable detection. Examples of such leuco dyes are disclosed in EP appl. no. 22184563.9 as well as applications claiming priority therefrom, which are incorporated herein by reference in their entirety (see, in particular, the Examples detailed therein). Leuco dyes which may be particularly useful include one or more of methyl green, basic fuchsin, acid fuchsin, crystal violet, malachite green or derivatives thereof. Alternatively or additionally, the test strip may comprise a rhodamine B—Cu dye which changes color in the presence of pyrophosphate. Utilization of such a rhodamine B—Cu dye can be implemented as described in PRADEEP Kumar, et al. Copper complex of a thienyl-hydrazone rhodamine derivative is a highly selective colorimetric sensor for pyrophosphate. Tetrahedron Letters. Volume 89, 2022, 153606, ISSN 0040-4039. Such a rhodamine B—Cu dye may utilized in solution or immobilized on a test strip. Both types of functionalization allow for a simple color change detection method of amplified DNA or RNA. Preferably, the device further comprises a heating arrangement comprising a heating element adjacent the at least one incubation chamber. Preferably, the heating element is mechanically attached and/or bonded to the housing. As such, the device may be a single-use nucleic amplification device including the heating element.

Preferably, the heating element is an electrical heating element. Preferably the heating element is a resistive-type heating element. The heating element may be formed on a printed circuit board (PCB). Alternatively, the heating element may be a chemical heating element, the heating element being configured to produce a chemical reaction and to heat the at least one incubation chamber to a target temperature by said chemical reaction.

Preferably, the amplification device further comprises at least one plug, for example a USB plug, for connecting to an electric power supply, preferably wherein the plug is provided in the housing.

Alternatively or additionally, the amplification device may comprise at least one cable for connecting to an electric power supply, preferably wherein the cable is plugged into or firmly attached to the housing.

Alternatively or additionally, The device may comprise at least one battery for providing energy to the heating element, preferably wherein the battery is received in the housing and/or attached to the housing.

Preferably the device comprises a microelectronic control element in electrical communication with the heating element. The microelectronic control element may be configured to control a supply of electrical energy to the heating element (e.g., electrical energy stemming from the plug, cable, or battery). The microelectronic control element may be configured to supply electric energy to the heating element in predetermined time intervals and/or in accordance with a temperature signal.

Preferably, the device comprises one or more temperature sensors, such as thermistors, preferably at least one temperature sensor for each incubation chamber. Each of the one or more temperature sensors may output a signal to the microelectronic control element.

Preferably, the device is configured to determine autonomously that the bodily fluid sample has been introduced into the device and/or received in the one or more incubation chambers. This may further facilitate use of the device in home environments.

For example, an electrical circuit between a power supply of the heating arrangement and the heating element is open in the absence of sample within the amplification device, and is closed upon the presence of sample within the amplification device. Preferably, the sample acts as an electrical conductor to close the electrical circuit, e.g. to close an electrical connection between the battery and the heating element. Such arrangement may be particularly advantageous in that the battery is not required to power a sensor and/or a microelectronic control element until the sample is present.

Preferably, the device comprises a plurality of incubation chambers, wherein the heating arrangement is configured to heat each of the incubation chambers to a respective target temperature, wherein the target temperature differs between the incubation chambers. This may facilitate amplification of different DNA/RNA sequences for detecting different illnesses since the preferred temperatures for amplification may depend on the respective sequence, primer and/or other reagents.

Preferably, one or more incubation chambers that are to be heated to a higher target temperature are arranged between incubation chambers that are to be heated to a lower target temperature. This may simplify the design and/or the control of the heating element.

Preferably, the heating arrangement is configured to provide a maximum temperature of 75° C., a maximum temperature of 70° C., or a maximum temperature of 65° C.

Preferably, the heating arrangement is configured to provide a minimum temperature of 50° C., 55° C., or 60° C.

According to a further aspect, the present disclosure is also directed to a nucleic acid amplification system comprising the amplification device as previously described and an external electronic device with a camera. The external device may be a handheld device such as a mobile phone or 6 a tablet and be configured to capture an image of the incubation and detection section and evaluate whether amplification of nucleic acids has been detected.

The external device may be connected to a remote server, e.g. through a wireless connection and/or a cellular network. When reference is made hereinafter to the external device being “configured” or programmed” in a certain manner, it should be understood that some of the respective processes may also be performed on the server.

Preferably the external device (e.g., the device as such or in conjunction with the server) is configured to detect a color of a test composition, e.g. the color of a test strip, of a test powder, or of a test liquid. Preferably, the device is configured to determine based on the detected color whether DNA amplification has occurred. In other words, the external device may be configured to identify whether the amplification device has detected the presence of one or more illnesses in the sample. The external device may be configured to output to the user the result identified.

For example, the user may be directed to take an image of the amplification device (e. g, in the instructions for use and/or via an application running on the external device), preferably an image of the amplification device wherein several of the chambers in which the test composition is provided are visible (e.g., several or all of the detection chambers and/or several or all of the incubation chambers). The external device may then determine (either the device as such or in conjunction with the server) whether and in which of the one or more detection chambers a change in color indicates the presence of the respective illness(es).

The external device (e.g., the device as such or in conjunction with the server) may be configured to modify parameters of the image to improve identification of the color change indicative of the illness(es). For example, the external device (e.g., the device as such or in conjunction with the server) may be configured to modify the hue, saturation, brightness, and/or contrast of the image.

More preferably the external device is programmed to detect a blue color, in particular a blue color of a leuco dye; and/or the external device is programmed to detect a pink color, in particular a pink color of a rhodamine B—Cu dye. Without wanting to be bound by theory, it has been found by the inventors that such colors may be detected well in an automated fashion by such external devices.

Preferably the housing of the amplification device further comprises at least one identification marker, more preferably a QR code. The external device may be programmed to detect the at least one identification marker and identify the orientation of the housing based thereon. The external device may be configured to correlate each of the chambers to a respective illness based on the identified orientation. The external device may be configured to indicate to the user (e.g., via the application) which illness(es) has/have been identified based on this correlation. Preferably, the user manual and/or the application directs the user to take the above-mentioned image of the chambers such that the identification marker is shown therein.

The above-mentioned identification marker (e.g., the QR code) could also be employed to identify via the external device whether the test is authentic (e.g., by including a unique code) and/or to identify the serial number and/or the production batch number of the amplification device.

According to a further aspect, the present disclosure is also directed to a method for testing a bodily fluid sample for illness. The method comprises the steps of providing a liquid sample derived from a bodily fluid; providing the amplification device or the amplification system according to any of the previous aspects, combining at least one nucleic acid amplification composition including a primer configured to amplify nucleic acids indicative of an illness with the liquid sample to form at least one amplification mixture in the amplification device; incubating the amplification mixture in the amplification device at a temperature which enables nucleic acid amplification; and detecting the presence of amplified nucleic acids within the amplified sample, preferably via a color change, more preferably by identifying such color change in an image taken with the external device.

According to a further aspect, the present disclosure is directed to a microfluidic mixing element, wherein the mixing element comprises multiple channels being fluidly connected in series with multiple mixing chambers, the channels and the mixing chambers being arranged in an alternating configuration, preferably wherein the channels are configured to provide laminar flow for a liquid sample and the mixing chambers are designed to provide turbulent flow for the liquid sample.

### BRIEF DESCRIPTION OF THE DRAWINGS

The subject matter of the present disclosure will be explained in more detail in the following text with reference to preferred exemplary and non-limiting embodiments which are illustrated in the attached drawings.

FIG. 1 illustrates a perspective view of an embodiment of the nucleic acid amplification device;

FIG. 2 is a view from above of an embodiment of the nucleic acid amplification device;

FIG. 3 illustrates cross-sections through the device as shown in FIG. 2;

FIG. 4 illustrates a bottom view of a second layer of the housing of FIG. 1;

FIG. 5 illustrates cross-sections through the device as shown in FIG. 4;

FIG. 6 depicts a further embodiment of the nucleic acid amplification device;

FIG. 7 is an illustration of one type of heating arrangement of the nucleic acid amplification device;

FIG. 8 is a heat map of the temperature profile provided to incubation chambers by the heating arrangement;

FIG. 9 is a magnified heat map image of the incubation chambers;

FIG. 10 depicts a further embodiment of the nucleic acid amplification device;

FIG. 11 illustrates a view from above of the amplification device of FIG. 10;

FIG. 12 illustrates a bottom view of the second layer of the view shown in FIG. 11;

FIG. 13 illustrates cross-sections through the device as shown in FIG. 11;

FIG. 14 illustrates cross-sections through the device as shown in FIG. 12;

FIG. 15 depicts the embodiment of the device as shown in FIGS. 10-14 including a heating arrangement; and

FIG. 16 depicts a bottom view of the device of FIG. 15.

These figures disclose embodiments of the present disclosure for illustrational purposes only. In particular, the disclosure provided by the figures and description is not meant to limit the scope of protection conferred by the present disclosure.

### DETAILED DESCRIPTION

Before the present disclosure is described in greater detail, it is to be understood that it is not limited to particular embodiments described, and as such can, of course, vary. Alternative embodiments of the structures and methods illustrated herein can be employed without departing from the principles of the disclosure described herein. It is also to be understood that the terminology used herein is for the purpose of describing particular embodiments only, and is not intended to be limiting, since the scope of the present disclosure will be limited only by the appended claims.

It is to be understood that wherever numerical ranges are provided/disclosed herein, all values and subranges encompassed by the respective numerical range are meant to be encompassed within the scope of the present disclosure. Accordingly, the present disclosure specifically and individually relates to each value that falls within a numerical range disclosed herein, as well as each subrange encompassed by a numerical range disclosed herein.

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted. In case of conflict between the plain meaning and the provided definitions, the provided definitions are to be used.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “optional”, “optionally” and “may” denote that the indicated feature may be present but can also be absent. Whenever the term “optional”, “optionally” or “may” is used, the present disclosure specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the present disclosure specifically relates to both possibilities, i.e., that the corresponding component is present (contained in the composition) or that the corresponding component is absent from the composition.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The term “about” can mean within an acceptable error range for the particular value as determined by one of ordinary skill in the art, which will depend in part on how the value is measured or determined, e.g., the limitations of the measurement system. For example, “about” can mean plus or minus 5%, 10%, or 20%, per the practice in the art. In certain embodiments, the term “about” refers to being within manufacturing tolerance levels as known by one of ordinary skill in the art (e.g., AS 1163, EN 10219, ASTM A500 or G 3444/G3466 tolerance level standards). In case of doubt, encompassed within the term “about” are numbers that are insignificantly different from the stated number.

Where ranges and/or subranges of values are provided, the ranges and/or subranges can include the endpoints of the ranges and/or subranges.

In addition, it is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

In the following detailed examples of the present disclosure, the device will be discussed in the context of testing for sexually transmitted infections (STIs). However, it will be apparent to those skilled in the art that the device could also be employed in testing for other types of illnesses, including urinary tract infections, screening for cancer, etc. Additionally, the nucleic acid amplification device as described herein may also be referred to as a cassette.

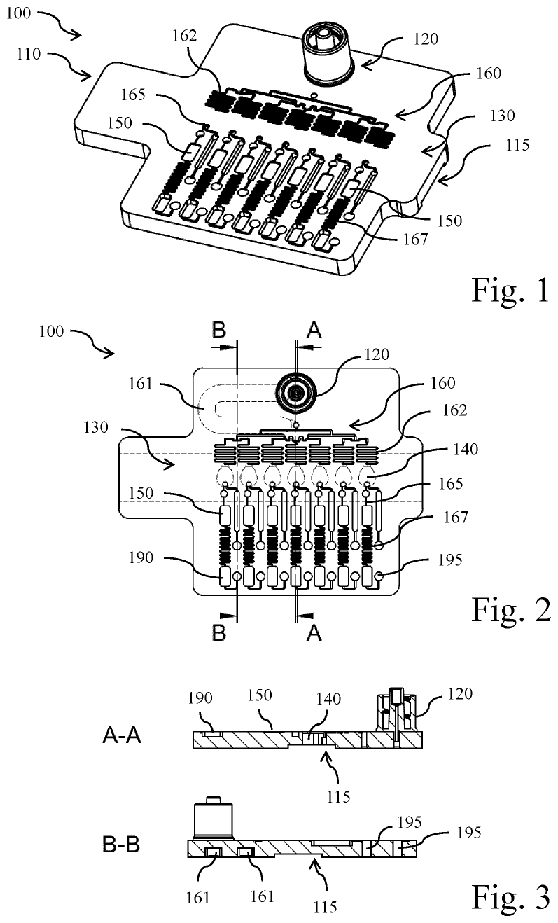

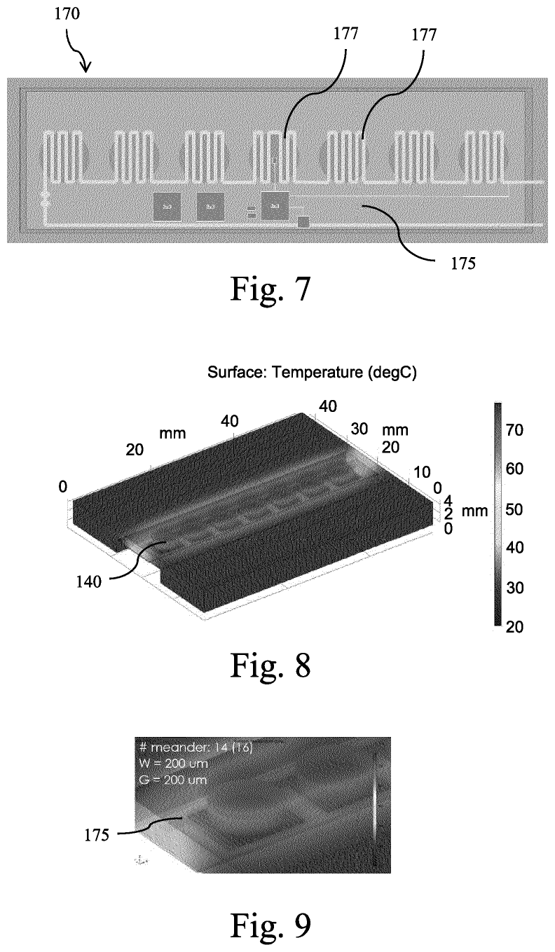

FIG. 1 depicts an embodiment of the nucleic acid amplification device 100 (or cassette) according to the present disclosure. The device includes a housing 110, which functions as a platform for the microfluidic channels and reservoirs as well as the heating components and the inlets of the device. One portion of the housing 110 functions as the inlet port 120 through which a liquid sample can be provided into the cassette. Another portion of the cassette functions as the incubation and detection section 130 having one or more incubation chambers 140. One or more first microfluidic conduits 160 connect to the inlet port 120, forming a liquid connection between the inlet port 120 and the incubation chamber 140. The nucleic acid amplification device 100 also comprises a plurality of nucleic acid amplification compositions. These compositions may be located along the first microfluidic conduit 160 or in the incubation chambers 140. Positioned adjacent to the incubation chambers 140 is a heating arrangement 170 comprising a heating element 175 (not shown in FIG. 1, see FIG. 7).

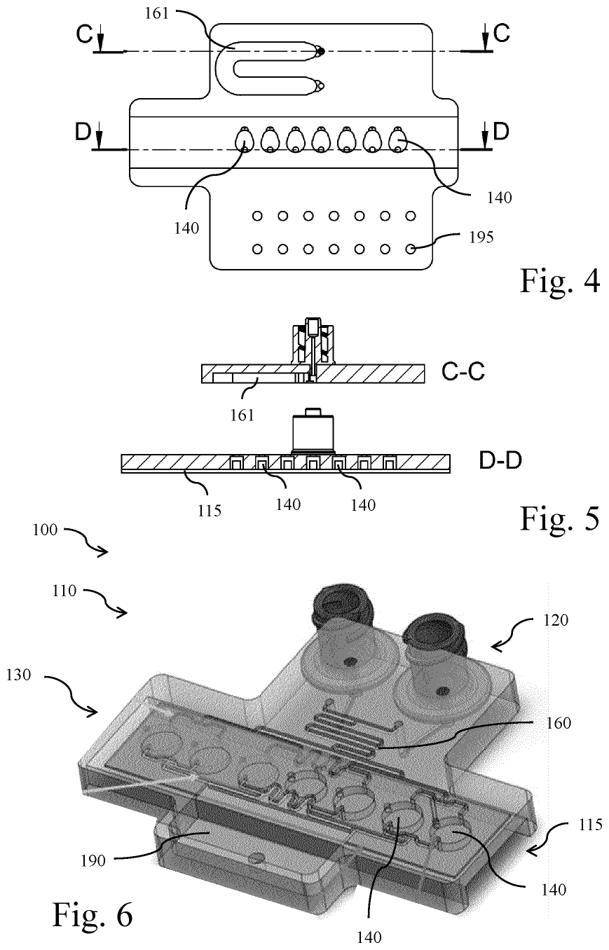

Design of the nucleic acid amplification device 100 is guided by the need for a simple, convenient, and private device for at home STD testing. As such, the housing 110 of the device may be light weight, easily produced, and ideally recyclable. The housing 110 in some embodiments may comprise a first layer and a second layer. Visible in FIG. 1 is the first layer, the second layer is shown in FIG. 4 and FIG. 2 provides a combined view of both layers. The second layer underlies the first layer. This first layer may be made from a rigid plastic material, such as polycarbonate, polypropylene or PET. Preferably, the first layer is translucent, more preferably transparent. The second layer may be made from a softer material, such as silicone.

The inlet port 120 of the housing 110, as shown in FIG. 1 can optionally be formed as Luer Lock connection, a docking station for a further device, or a direct inlet adapted to receive a bodily fluid sample. Generally, the sample may be provided from any bodily fluid likely to contain identifiable genetic material. However, for some applications a provided urine sample or saliva sample may be most convenient. The sample provided is advantageously concentrated such that any cellular material present therein can be more easily extracted. Such a sample preparation system is described, for example, in the EP application no. 22 179 963 (as well as any applications claiming priority therefrom), the entire contents of which are incorporated by reference herein. As such, the sample may comprise an amount of prepared DNA/RNA available in the form of a lysate. The sample preparation system can be connected or docked at the inlet port 120 then the sample can be transferred from the sample preparation system into the cassette.

In further embodiments it is conceived that a sample preparation unit, such as described in EP 22 179 963 may be physically incorporated within the cassette and configured to provide the liquid sample through the inlet port 120. Thus, such a combined device would prepare the fluid sample (in particular, concentrate the cellular material), amplify nucleic acids derived therefrom, and then enable detection of amplified nucleic acids all within one device.

After passing through the inlet port 120, the liquid sample passes through the one or more first microfluidic conduits 160. A portion of the first conduits 160 may be constructed such that an equal amount of the liquid reaches each of the incubation chambers 140 connected thereto. This can be accomplished as is known in the art, e.g. by varying the length or width of each of the first microfluidic conduits 160, e.g. to provide “more resistance” within conduits leading to incubation chambers 140 closer to the inlet and “less resistance” in conduits leading to incubation chamber 140 positioned further away from the inlet. The first microfluidic conduits 160 may also be provided with one or more one-way valves to prevent backflow of the liquid sample, e.g. at least one valve per microfluidic conduit 160. In the embodiment shown in FIG. 1 the first microfluidic conduits 160 comprise a common first leg 161 and a second leg 162, wherein the first leg 161 provides a reservoir for the liquid sample received from the inlet and the second leg 162 divides and equally distributes the liquid sample between each of the incubation chambers 140. The second leg 162 may also act as a flow resistor which enables control of the flow rate of the sample.

The incubation and detection section 130 of the cassette includes at least one, but preferably a plurality of incubation chamber 140. Each incubation chamber 140 may have a volume of 25 μL.

The incubation and detection section 130 may comprise at least 2, 3, or 5 incubation chambers 140 and/or between 2 and 12, between 2 and 7, between 3 and 7, or 5 incubation chambers 140.

The incubation chambers 140, which may also alternatively be known as amplification chambers, provide a volume in which DNA/RNA amplification can take place. Amplification of nucleic acids within the device 100 preferably takes place using loop-mediated isothermal amplification (LAMP) or reverse transcription loop-mediated isothermal amplification (RT-LAMP), which are well established techniques. LAMP (and RT-LAMP) require specific temperatures to be maintained within the reaction volume. As such, the incubation chambers 140 are positioned adjacent to a heating arrangement 170. Specific implementations of the heating arrangement 170 will be discussed further with respect to FIG. 7 to FIG. 9.

Nucleic acid amplification compositions are positioned within each incubation chamber 140, or along the first microfluidic conduits 160 which enable nucleic acid amplification. Said compositions are used to target DNA or RNA specific to one or more illnesses. The specificity of each composition is derived from the one or more primers, i.e. oligonucleotides, which are specific to a certain sequence of DNA/RNA present within bodily fluids in the event of an illness. The nucleic acid amplification composition may further include suitable buffers, enzymes, DNA polymerase, reverse transcriptase, salts, deoxyribonucleotides, and/or ribonucleotides.

In some embodiments the nucleic acid amplification compositions are provided in the form of lyophilized pellets. As such the nucleic acid amplification composition is freeze-dried and provided within the cassette during assembly. Such pellets are configured to dissolve upon contact with a liquid sample. Advantageously, these pellets are relatively stable and may be maintained at room temperatures and over long time periods. In some cases the lyophilized pellets may include lyoprotectants such as sugars and polyalcohols and/or cryoprotectants which help to preserve the function of enzymes contained therein.

Alternatively, the nucleic acid amplification compositions may be provided in the form of a sphere or as a powder. Any of these options provides for convenient storage of the nucleic acid amplification compositions to enable long-term storage, retail sale, shipping and home use of the device.

In some traditional detection schemes, labeling of amplified DNA is performed simultaneously during incubation. As such, the nucleic acid amplification compositions may further include labeling microparticles or fluorescent sequences which can be detected using fluorescence microscopy. Alternatively or additionally, presence of amplified DNA and/or RNA may be assessed by measuring the turbidity of the sample in the incubation chamber before and/or after amplification. Further detection arrangements and methods will also be discussed below.

Preferred embodiments of the amplification device 100 include at least one detection chamber 150 within the incubation and detection section 130 of the device. The detection chamber 150 is fluidly connected to the incubation chamber 140 through one or more second microfluidic conduits 165.

As depicted in FIG. 1 the housing 110 includes a first layer which includes the inlet port 120 and some features of the incubation and detection section 130. FIG. 4 illustrates an embodiment of the second layer, which includes a portion 161 of the first microfluidic conduit 160 leading from the inlet port 120 to a second portion 162 of the first microfluidic conduit 160 which is visible in FIG. 1. Further visible within the FIG. 4 are seven incubation chambers 140. These incubation chambers 140 are positioned over a pocket 115, or cavity within the housing 110, which is configured to receive the heating element 175 (see, e.g., FIG. 3). This pocket 115 can also be observed in cross-section D-D of FIG. 5 in which a narrow, empty cavity underlies the length of the device including each of the incubation chambers 140. In FIG. 2 a top-down view of the amplification device 100 is presented which includes the first layer and the second layer, wherein the second layer is represented by dashed-line features.

In the embodiment of FIG. 1, after incubation has taken place within the incubation chamber(s) 140 the sample is further moved to the detection chambers 150. This includes passing the liquid amplified mixture out of the incubation chamber(s) 140 through the one or more second fluid conduits 165 and into the detection chamber(s) 150. In some embodiments the number of detection chambers 150 may correspond to the number of incubation chambers 140, with a separate second fluid conduit 165 for each pair. The second microfluidic conduit 165 may include another flow resister. The second microfluidic fluid conduit 165 may also comprise a further one-way valve to prevent backflow of fluids. Said one-way valves may also be simple capillary valves known in the art of microfluidics.

The detection chambers 150 as shown in FIG. 1 and FIG. 2 act as reservoirs which enable the detection of amplified nucleic acids. Separate incubation chambers 140 and detection chambers 150, as shown in the embodiment of FIG. 2 facilitate certain detection methods, such as employing a functionalized test strip which is pre-positioned within the detection chambers 150. When the amplified sample mixture contacts the test strip a change in color may indicate the presence of amplified nucleic acids.

One particularly advantageous example of such a functionalized test strip employs a test strip of cellulose acetate functionalized with a leuco dye. Such a method of colorimetric nucleic acid detection is described in detail by Applicant's further EP appl. no. 22184563.9 as well as any subsequent patent applications claiming priority therefrom, which are incorporated herein by reference in their entirety (see, in particular, the Examples detailed therein). Therein described is the form and function of the leuco dye and how, upon contact with a threshold amount of nucleic acid, the leuco dye changes color. In such a configuration the functionalized test strips are not specific to any particular type of amplified nucleic acids but rather indicate the amount of DNA and/or RNA present in the liquid. Instead, the specific primers provided to each incubation chamber 140 or first microfluidic conduit 160 determine which detection chamber 150 is specific for the detection of which illness. This method of detection is particularly advantageous, in that detection strips do not need to be individually tailored, and detection can take place with a visual readout of the detection chambers 150.

In the interest of ease of use, the nucleic acid amplification device 100 is configured to be simple and user-friendly. As such, operation of the amplification device 100 may advantageously take place within the scope of an amplification system which further comprises an external electronic device, such as, for example, a handheld device, a mobile phone, or a tablet (not shown). The external electronic device may be configured to include a program (also referred to as an “application” herein) which guides the user through the operation of the device. The external device advantageously includes a camera, which can be utilized to take a photograph of the device after incubation and detection steps have been completed.

Optionally, the housing 110 of the device may include at least one identification marker. Such a marker on the housing 110 should be made detectable to the external device, and is, preferably, a QR code. The identification marker is configured to allow the external device to detect the orientation of the housing 110 relative to the camera and thereby determine the identities of each of the incubation chambers 140 and/or detection chambers 150. Cameras, particularly those in mobile devices, are very efficient at detecting the color blue. Moreover, the external device can be programmed to automatically increase the contrast and/or color value corresponding to the color of the test strip. The external device may be configured to modify, e.g., the hue, saturation and/or brightness of the image, e.g. to further accentuate the color blue.

As such, the external device is configured to detect whether a color change is present within each of the incubation chambers 140 and/or within each of the detection chambers 150. The external device can then provide a readout to the user regarding the results of the detection.

The identification marker provided on the housing 110 of the amplification device 100 may also convey other information, such as the batch number and/or serial number of the device and the external device may be programmed to extract this information.

In a further embodiment depicted in FIG. 6 the inlet port 120 comprises two docking ports, the first being for introduction of the prepared sample and the second being an additional inlet for a fluid that can be used to exert a force on the sample so as to assist the sample in moving through the cassette. In this instance, a user would first dock the sample preparation system at the first inlet and then using a pressure gradient created, for example, by a syringe, force the liquid sample out of the sample preparation system into the cassette. Then a second reservoir may be docked at the second inlet and using a pressure gradient created, for example, by a syringe, force the liquid, i.e. water or saline, out of the reservoir and into the cassette, thereby moving the liquid sample further through the cassette. Such a second docking port may be incorporated within any embodiment.

FIG. 6 includes a first microfluid conduit 160 leading from the inlet to a set of incubation chambers 140. In this embodiment, however, incubation and detection may both take place within the incubation chamber 140. Consequently, no second microfluidic channels 165 or detection chambers 150 are present within this embodiment. An overflow chamber 190 is, however, provided as an outlet for excess fluid sample. Smaller overflow chambers 190 (or a common overflow chamber as in FIG. 6) may similarly be provided within the first embodiment. Third microfluidic conduits 167 may be provided to fluidly connect the detection chambers 150 with the overflow chambers 190. Additionally, the microfluidic flow path may be provided with vents 195, particularly in connection with overflow chambers 190.

Detection methods which may be more suitable to detection directly within the incubation chamber 140, such as in FIG. 6, include the use of rhodamine B—Cu dye. Said dyes are pink in color and turn colorless in the presence of pyrophosphate. Such a color change can be observed by eye or alternatively be detected with the help of external device such as is described above.

Other methods for labeling/detection of amplified DNA and RNA which are known in the art include fluorescence detection which can be performed by labeling the copied nucleic acids with microparticles, such as oligonucleotide-conjugated quantum dots, or the incorporation of fluorescent dyes or fluorescently modified oligonucleotides. Additionally, detection may be performed using electrode detection, which measures the impedance across, for example, the liquid sample within the incubation or detection chambers 150, which changes in the presence of and relative to the amount of DNA/RNA.

FIG. 7 provides on example of one embodiment of the heating arrangement 170 which can be implemented within the amplification device 100. The heating arrangement 170 may be configured to be removable from the housing 110 of the amplification device 100. For example, the heating arrangement 170 may be configured to slot into a pocket 115 of the housing 110.

Alternatively, the heating arrangement 170 may be firmly connected to the housing 110. For example, the heating arrangement 170 may be mechanically attached and/or bonded to the housing 110 of the device. As such, the amplification device 100 may be configured to be disposable together with the heating arrangement 170. In other words, both the amplification device and its heating arrangement may be disposable and/or configured for single use.

The heating element 175 in this case includes a printed circuit board wherein heating is performed by resistance within the circuit. As can be seen in FIG. 7, certain heating portions 177 of the circuit may be provided, each of which preferably is configured to underly an individual incubation chamber 140. Power can be supplied to the heating element 175 by one or more batteries incorporated within the heating arrangement 170. Alternatively, the amplification device 100 can include at least one plug or cable, such as a USB plug or a wall plug for connecting to an electric power supply.

An alternative embodiment of the heating element 175 may be based on chemical heating, which using an exothermic chemical reaction is configured to heat the incubation chamber 140. In such a configuration the heating element 175 may still be configured to be removable from the housing 110 (e.g., to slot into the housing 110) and/or may be firmly connected to the housing, as described above.

In any configuration, the heating arrangement 170 may be configured to maintain the incubation chamber(s) 140 within the target range for amplification. This target range may have a maximum temperature of 75° C., preferably 70° C., and more preferably 65° C., and/or a minimum temperature of 50° C., preferably 55° C., more preferably 60° C. This target range may be pre-configured, in either the specifications of the resistive heating element 175 or the composition and distribution of the chemical heating element 175.

Some embodiments may further include a microelectronic control element as a part of the heating arrangement 170, wherein the control element is in communication with the heating element 175. Further, the heating arrangement 170 may include one or more temperatures sensors, such as thermistors, which monitor the temperature within the incubation chamber(s) 140. The control element may then use the information from the temperature sensor(s) to modulate the power supplied to the circuit and thereby regulate the temperature. Advantageously, each incubation chamber 140 is supplied with a separate temperature sensor.

As can be viewed in FIG. 8 and FIG. 9, the heating element 175 is configured to maintain each of the incubation chambers 140 within the target range. However, it can be envisioned that different incubation chambers 140 may need to be heated at different temperatures due to the particularities of the nucleic acids being amplified. Furthermore, the distribution of incubation chambers 140 and the heating element 175 as envisioned here, may provide the incubation chambers 140 which require being heated to higher temperature to be placed between other incubation chambers 140 which are to be heated to a lower temperature. In this way, some of the heat radiated away from an individual incubation chambers 140 may be utilized to provide extra heat to the neighboring incubation chamber 140.

One further advantage of the present disclosure is the provision of a heating arrangement 170 which is relatively simple and can be disposable. Again, this supports the use of the amplification device 100 by home users who are not healthcare professionals. In this aspect, automatic heating of the incubation chamber 140 without user intervention is highly advantageous. As such, the heating arrangement 170 may be configured such that the electrical circuit of the heating element 175 is open in the absence of a liquid sample and then closed upon provision of a liquid sample to the amplification device 100. One way in which this may be accomplished, is that the liquid sample may act as an electrical conductor, and when the liquid sample is provided to the amplification device 100, the liquid sample flows into a predetermined gap in the electrical circuit of the heating element 175. Therefore, the liquid sample acting as an electrical conductor closes the electrical gap in the circuit of the heating element 175, allowing the heating element 175 to begin heating the incubation chamber 140. Alternatively, the amplification device 100 may further be provided with a sample detection means configured to detect the presence of a liquid sample in the device and/or in the incubation chamber 140. The sample detection means then initiates the heating of the incubation chamber 140. The sample detection device may include at least one sensor, such a liquid sensor or conductance sensor, which triggers upon the introduction of a liquid sample. Alternatively, the amplification device 100 may be provided with a switch allowing the user to manually turn on and/or to manually turn off the heating element 175.

The present disclosure further includes a method for testing a bodily fluid sample for illness. The method comprises the steps of providing a liquid sample derived from a bodily fluid, combining at least one nucleic acid amplification composition including a primer configured to amplify nucleic acids indicative of an illness with the liquid sample to form at least one amplification mixture; incubating the amplification mixture at temperature which enables nucleic acid amplification, detecting the presence of amplified nucleic acids within the amplified sample. This method is advantageously performed using the nucleic acid amplification device 100 as described above.

The liquid sample provided to the device may come from a sample preparation unit as described above or may be provided directly into the amplification device 100. In the case of STI testing, urine and/or saliva are preferred testing fluids as they are easily provided and are often sites of infection for sexually transmitted diseases. Providing the sample may further include concentrating the cellular contents of the liquid sample. Other uses envisioned for the present method and system include testing for certain types of cancers, fungal, and protist infections. It is preferred that the amplification device 100 is configured to amplify nucleic acids of at least one of Chlamydia trachomatis, Neisseria gonorrhoeae, Mycoplasma genitalium or Trichomonas vaginalis.

In some preferred embodiments of the method, the liquid sample dissolves at least one lyophilized pellet, sphere or powder, wherein the pellet, sphere or powder comprises the nucleic acid amplification composition. This may include at least 2, at least 3, at least 5, or at least 7 individual pellets, spheres or powders, wherein each pellet, sphere or powder preferably comprises unique primers (i.e., preferably at least one primer relevant to a respective, different illness).

The amplification mixture is then amplified within the incubation chamber 140 at temperatures as described above. Incubation times may vary based on the specific implementation of the method. The incubation time may include at least 20 minutes, ideally 30 minutes. The incubation time may be maximally 1 hour, During amplification the target DNA/RNA sequences within the sample are amplified when present. Incubation is preferably performed automatically, without input from the user.

Following incubation, the device may automatically continue to the detection step. Alternatively, the user may be provided a notification, through, for example, an external device, that incubation has been accomplished and next steps should be taken.

As described above, detection may take place directly within the incubation chamber 140 or within separate detection chambers 150. Detection may be a simple visual inspection of the incubation chambers 140 or the detection chambers 150 to identify whether a color change has taken place. Alternatively, the user may use an external device to photograph the amplification device 100 and the external device is configured to present the detection results to the user.

After detection has been read out, the amplification device 100 may be disposed of or preferably recycled. As such, the present method is suitable for carrying out on a single use and/or disposable amplification device 100 as described above.

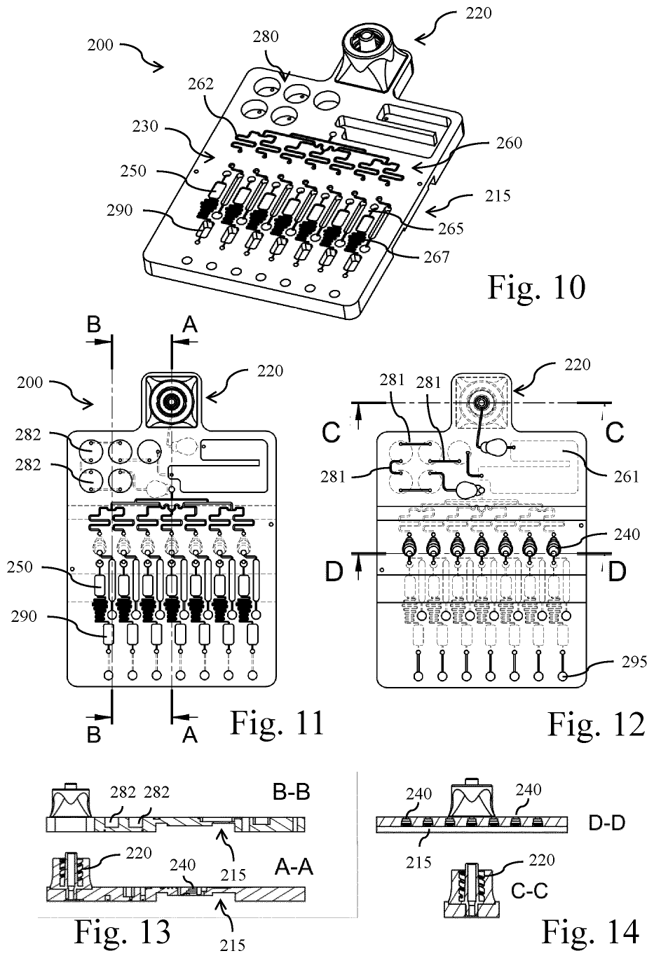

In FIGS. 10-14 a further embodiment of the amplification device 200 is illustrated. The features of this embodiment correspond generally to the previously described embodiments as indicated by the corresponding reference signs (shifted by 100). Reference is made to the description for the corresponding elements above in order to avoid repetitions.

The embodiment of FIG. 10 further includes a mixing element 280 within the first microfluidic conduits 260, preferably in between the first leg 261 and the second leg 262. The mixing element 280 comprises at least one narrow channel 281 and at least one mixing chamber 282. For example, the mixing element 280 may contain a plurality of narrow channels 281 and a plurality of mixing chambers 282, wherein the narrow channels 281 and the mixing chambers 282 are arranged in an alternating configuration along the direction of fluid flow within the amplification device 200. This arrangement is particularly advantageous in implementations of the amplification cassette wherein the liquid sample needs to be pre-mixed before entering the incubation and detection section 230 of the device 200. The mixing element 280 may allow for thorough mixing such that a cell lysate sample is homogeneously distributed within the buffer. Alternatively, the mixing chambers 282 may be provided with pre-processing agents for mixing with the liquid sample.

Each reservoir 282 preferably is connected to an inlet channel 281 forming an inlet through which fluid is delivered into the reservoir 282 and an outlet channel 281 forming an outlet through which fluid flows out of the respective reservoir 282. The outlet channel 281 may connect to a following reservoir 282.

The inlet and/or outlet may be formed along a bottom surface of the reservoir 282. In other words, the flow from the inlet may be directed upwards and/or the flow into the outlet may be directed downwards.

The mixing element 280 promotes mixing of the sample fluids by alternating fluid flow though narrower channels 281 and the comparatively larger reservoirs defined by the mixing chambers 282. For example, the narrow channels may have a cross-sectional area of 1.0 mm² or less, preferably 0.5 mm² or less, more preferably 0.3 mm² or less as measured in a cross section taken perpendicularly to the flow path through the respective channel. The reservoirs may have a cross-sectional area of at least 20 mm² , preferably at least 25 mm² , more preferably at least 28 mm² as measured in a horizontal cross section.

In other words, the reservoir 282 may have a cross section that is at least X times, preferably at least X times, the cross section of the inlet and/or the cross section of the outlet, each measured in a horizontal cross section.

Flow patterns within the mixing element 280 are defined by Reynolds number, wherein at a low Reynold's number, generally less than 1000 Re, the flow is laminar. At a relatively high Reynold's number, generally greater than 2300 Re, the flow is turbulent. This may be further defined as between 500 Re and 1000 Re for the narrow channels 281 and/or between 2300 Re and 3000 Re for the mixing chambers 282. In other words, the narrow channels 281 may be designed to have a Reynolds number within the laminar flow regime and the mixing chambers 282 may be designed to have a Reynolds number within the turbulent flow regime. These regimes may also be defined by a width of the narrow channel 281 in comparison with a width of the mixing chamber 282 in the direction of fluid flow, wherein the width of the mixing chamber 282 is at least four times, five times, or six times the width of the narrow channel 281.

It should also be noted that the entry points and exit point within a mixing chamber 282 should be separated from one another to prevent any unmixed flow from leaking through. The walls of the mixing chamber 282 may be curved and/or rounded to promote the turbulent mixing of fluid therewithin. The mixing chamber 282 may have a cylindrical form.

It is also envisaged that such a mixing element 280 may be incorporated in other locations on the amplification device 2000, such as between the incubation chambers 240 and the detection chambers 250.

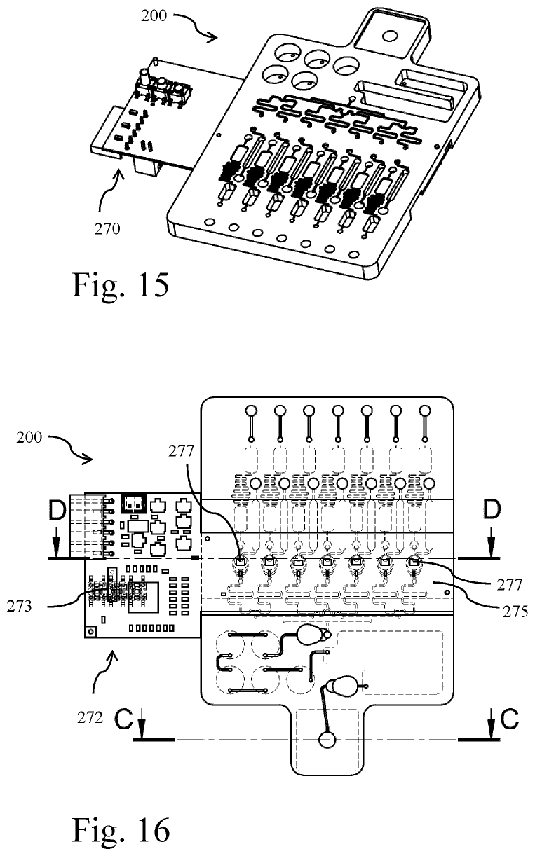

FIG. 15 and FIG. 16 illustrate an embodiment of the amplification device 200 including an inserted heating arrangement 270. The heating arrangement 270 in this embodiment comprises a control portion 272 and a heating portion 275, wherein the heating arrangement 270 is inserted into the pocket 215 of the amplification device 200. The control portion 272 comprises a circuit board 273 which is in electrical connection with the heating elements 277. The heating portion 275 comprises individual heating elements 277, each of which is positioned directly underneath each of the incubation chambers 240 of the amplification device 200. Each of the heating elements 277 in this embodiment are connected to the circuit board 273 and may be individually controlled by the control portion 272. Each heating element 277 may be heated to the same temperature, or each may be heated to a individual temperature. The heating arrangement 270 may be bonded to the amplification device 200, such that together they form one unit. The heating arrangement 270 and the amplification device 200 may be disposable and/or configured for single use.

While the invention(s) has been illustrated and described in detail in the drawings and foregoing description, such illustration and description are to be considered illustrative or exemplary and non-restrictive; the invention(s) is thus not limited to the disclosed embodiments. Variations to the disclosed embodiments can be understood and effected by those skilled in the art and practicing the claimed invention(s), from a study of the drawings, the disclosure, and the appended claims.

The following are preferred aspects of the present disclosure:
1. A nucleic acid amplification device for testing a bodily fluid sample for illness, the device comprising a housing comprising:
   an inlet port for receiving a sample derived from a bodily fluid;
   an incubation and detection section configured to enable the amplification and the detection of nucleic acids, the section comprising at least one incubation chamber;
   one or more first microfluidic conduit connecting the inlet port with each of the one or more incubation chambers;
   at least one nucleic acid amplification composition including at least one primer configured to amplify nucleic acids indicative of an illness, each of the nucleic acid amplification compositions being located along each of the first microfluidic conduits and/or in each incubation chamber; and
   a heating arrangement comprising a heating element adjacent the at least one incubation chamber.
2. The amplification device according to aspect 1, wherein the incubation and detection section further comprises at least one detection chamber configured to enable the detection of amplified nucleic acids, and the incubation and detection section further comprises one or more second microfluidic conduits connecting each of the incubation chambers with a respective detection chamber.
3. The amplification device according to aspect 2, wherein the device comprises a plurality of detection chambers, preferably a number of detection chambers corresponding to the number of incubation chambers, wherein each incubation chamber is connected through one of the second microfluidic conduits to a corresponding detection chamber.
4. The amplification device according to aspect 2 or aspect 3, wherein each of the second microfluidic conduits comprises a one-way valve for preventing flow from the detection chamber towards the incubation chamber.
5. The amplification device according to any of the previous aspects, wherein the device is a single-use device and/or a single-use amplification cassette.
6. The amplification device according to any of the previous aspects, wherein the device comprises a plurality of incubation chambers, preferably wherein the device comprises at least 2, at least 3 or at least 5 incubation chambers and/or wherein the device comprises between 2 and 12 incubation chambers, preferably between 2 and 7, more preferably between 3 and 7, most preferably 5 incubation chambers.
7. The amplification device according to any of the previous aspects, wherein the nucleic acid amplification compositions are provided in the form of at least one lyophilized pellet, at least one sphere, or as a powder; preferably wherein the amplification compositions are provided in the form of a plurality of lyophilized pellets, spheres, or powders.
8. The amplification device according to aspect 7, wherein the device is configured to receive a sample that is a liquid suspension configured to dissolve the lyophilized pellet, sphere or powder on contact.
9. The amplification device according to aspect 7 or aspect 8, wherein the device comprises a plurality of incubation chambers and a plurality of first microfluidic conduits, each of the incubation chambers being connected to the inlet port by a respective first microfluidic duct, and wherein a lyophilized pellet, sphere or powder is arranged in each of the incubation chambers or along the respective first microfluidic duct.
10. The amplification device according to any of aspects 7-9, wherein the lyophilized pellet, sphere or powder comprises a nucleic acid amplification primer configured to amplify nucleic acids indicative of at least one illness.
11. The amplification device according to any of aspects 7-10, wherein the pellet, sphere or powder further comprises an enzyme, lyoprotectant and/or a cryoprotectant.
12. The amplification device according to any of the previous aspects, wherein each nucleic acid amplification composition comprises a different set of primers configured to amplify nucleic acids indicative of a different illness.
13. The amplification device according to any of the previous aspects, wherein the primers are configured to amplify nucleic acids of at least one of Chlamydia trachomatis, Neisseria gonorrhoeae, Mycoplasma genitalium or Trichomonas vaginalis.
14. The amplification device according to any of the previous aspects, wherein each of the first microfluidic conduits comprises a one-way valve for preventing flow from the incubation chamber towards the inlet port.
15. The amplification device according to any of the previous aspects, wherein the heating element is an electrical heating element.
16. The amplification device according to any of the previous aspects, wherein the heating element is a resistive-type heating element.
17. The amplification device according to aspect 15 or aspect 16, wherein the heating element is formed on a printed circuit board (PCB).
18. The amplification device according to any of the previous aspects, wherein the amplification device further comprises at least one battery for providing energy to the heating element, preferably wherein the battery is received in the housing and/or attached to the housing.
19. The amplification device according to any of the previous aspects, wherein the amplification device further comprises
   at least one plug, for example a USB plug, for connecting to an electric power supply, preferably wherein the plug is provided in the housing; and/or
   at least one cable for connecting to an electric power supply, preferably wherein the cable is plugged into or firmly attached to the housing.
20. The amplification device according to any of the previous aspects, wherein the heating element is a chemical heating element, the heating element being configured to produce a chemical reaction and to heat the at least one incubation chamber to a target temperature by said chemical reaction.
21. The amplification device according to any of the previous aspects, wherein the heating arrangement is configured to provide a maximum temperature of 75° C., preferably a maximum temperature of 70° C., more preferably a maximum temperature of 65° C.
22. The amplification device according to any of the previous aspects, wherein the heating arrangement is configured to provide a minimum temperature of 50° C., preferably 55° C., more preferably at least 60° C.
23. The amplification device according to any of the previous aspects, further comprising a microelectronic control element in electrical communication with the heating element.
24. The amplification device according to any of the previous aspects, further comprising one or more temperature sensors, such as thermistors, preferably at least one temperature sensor for each incubation chamber.
25. The amplification device according to any of the previous aspects, wherein the heating element is removable from the amplification device.
26. The amplification device according to any of the previous aspects, wherein the housing comprises a holder or pocket for receiving the heating element therein.
27. The amplification device according to any of the previous aspects, wherein the heating element is mechanically attached and/or bonded to the housing.
28. The amplification device according to any of the previous aspects, further comprising a sample detection means for detecting presence of a sample in the device, preferably a presence of sample in the incubation chamber.
29. The amplification device according to aspect 28, wherein the sample detection means is configured to initiate heating by the heating arrangement when the presence of a sample is detected.
30. The amplification device according to aspect 28 or 29, wherein the sample detection means comprises at least one sensor.
31. The amplification device according to any of the previous aspects, wherein an electrical circuit between a battery of the heating arrangement and the heating element is open in the absence of sample within the amplification device, and wherein the electrical circuit is closed upon the presence of sample within the amplification device.
32. The amplification device according to aspect 31, wherein the sample acts as an electrical conductor to close the electrical connection between the battery and the heating element.
33. The amplification device according to any of the previous aspects, wherein the device comprises a plurality of incubation chambers, wherein the heating arrangement is configured to heat each of the incubation chambers to a respective target temperature, wherein the target temperature differs between the incubation chambers.
34. The amplification device according to aspect 33, wherein one or more incubation chambers that are to be heated to a higher target temperature are arranged between incubation chambers that are to be heated to a lower target temperature.
35. The amplification device according to any of the previous aspects, wherein the incubation and detection section comprises at least one test strip, wherein the test strip has been functionalized or adsorbed with a dye configured for colorimetric detection of amplified nucleic acids such as DNA or RNA, preferably wherein the dye is a leuco dye or a rhodamine B—Cu dye.
36. The amplification device according to aspect 35, wherein the test strip is not specific to the one or more amplicons replicated in the one or more incubation chambers.
37. The amplification device according to aspect 35 or aspect 36, wherein the test strip is configured to change in color upon presence of a threshold amount of DNA.
38. The amplification device according to any of aspects 35-37, wherein the test strip comprises a leuco dye, preferably a leuco dye configured to turn to a blue color to enable detection.
39. The amplification device according to any of aspects 33-38, wherein the test strip comprises a rhodamine B—Cu dye which changes color in the presence of pyrophosphate.
40. The amplification device according to any of the previous aspects, wherein the inlet port comprises a docking station for connecting a sample preparation unit to the amplification device or wherein the amplification device further comprises a sample preparation section which is fluidly connected to the inlet port.
41. The amplification device according to any of the previous aspects, wherein the device is configured to receive a sample derived from a bodily fluid, preferably wherein the bodily fluid is urine and/or saliva.
42. A nucleic acid amplification system comprising:
   the amplification device of any of aspects 1 to 41; and
   an external electronic device with a camera, preferably a handheld device such as a mobile phone or a tablet, the external device being configured to capture an image of the incubation and detection section and evaluate whether amplification of nucleic acids has been detected.
43. The amplification system according to aspect 42, wherein the external device is programmed to detect a color of a test strip.
44. The amplification system according to aspect 42 or aspect 43, wherein the external device is programmed to detect a blue color, in particular a blue color of a leuco dye; and/or the external device is programmed to detect a pink color, in particular a pink color of a rhodamine B—Cu dye.
45. The amplification system according to any of aspects 42-44, wherein the external device is programmed to increase in the image the contrast and/or a color value corresponding to the color of the test strip.
46. The amplification system according to any of aspects 42-45, wherein the housing of the amplification device further comprises at least one identification marker, preferably a QR code, wherein the external device is programmed to detect the at least one identification marker and interpret the orientation of the housing based thereon.
47. The amplification system according to aspect 46, wherein the identification marker further comprises information such as the serial number and/or batch number of the amplification device, and wherein the external device is programmed to extract this information from the identification marker.
48. The amplification system according to any of aspects 42-47, wherein the external device is programmed to display results of the evaluation to a user.
49. The amplification system according to any of aspects 42-48, wherein the external device is programmed to provide instructions to a user how to operate the amplification system.
50. A method for testing a bodily fluid sample for illness, the method comprising the steps of:
   providing a liquid sample derived from a bodily fluid;
   combining at least one nucleic acid amplification composition including at least one primer configured to amplify nucleic acids indicative of an illness with the liquid sample to form at least one amplification mixture;
   incubating the amplification mixture at temperature which enables nucleic acid amplification;
   detecting the presence of amplified nucleic acids within the amplified sample.
51. The method of aspect 50, wherein combining the at least one nucleic acid amplification composition with the liquid sample comprises dissolving at least one lyophilized pellet, sphere or powder using the liquid sample, wherein the lyophilized pellet, sphere or powder comprises a nucleic acid amplification primer configured to amplify nucleic acids indicative of at least one illness.
52. The method of aspect 50 or aspect 51, wherein the method comprises at least three lyophilized pellets, spheres or powders, wherein each pellet, sphere or powder is situated in an individual incubation chamber.
53. The method of any of aspects 50-52, further comprising the step of reading out the result of the amplification detection using an external device configured to read out the detection result.
54. The method of any of aspects 50-53, wherein providing a liquid sample further comprises concentrating the cellular components of the bodily fluid.
55. The method of any of aspects 50-54, wherein incubating the amplification mixture further comprises maintaining the amplification mixture at a maximum temperature of 75° C., preferably 70° C., more preferably 65° C.; and/or at a minimum temperature of 50° C., preferably 55° C., more preferably at least 60° C.
56. The method of any of aspects 50-55, further comprising the step of providing instructions to a user for carrying out the method, the instructions being provided on an external device.
57. The method of any of aspects 50-56, wherein detection of amplified nucleic acids is performed using a leuco dye configured to change to a blue color when in contact with a threshold amount of nucleic acids and/or wherein the detection of amplified nucleic acids is performed using a rhodamine B—Cu dye configured to change from a pink color to colorless when in contact with a threshold amount of pyrophosphate.
58. The method of any of aspects 50-57, wherein the method is carried out by a user, preferably wherein the user is not a healthcare professional.
59. The method of any of aspects 50-58, wherein the method is carried out in a single-use device and/or a single-use amplification cassette.
60. A microfluidic mixing element, wherein the mixing element comprises multiple channels being fluidly connected in series with multiple mixing chambers, the channels and the mixing chambers in an alternating configuration, preferably wherein the channels are configured to provide laminar flow for a liquid sample and the mixing chambers are designed to provide turbulent flow for the liquid sample.
61. The microfluidic mixing element according to aspect 60, wherein the narrow channel is configured to provide a Reynolds number lower than 1000 Re, preferably between 500 Re and 1000 Re for the liquid sample.
62. The microfluidic mixing element according to aspect 60 or aspect 61, wherein the mixing chamber is configured to provide a Reynolds number greater than 2300 Re, preferably between 2300 Re and 3000 Re for the liquid sample.
63. The microfluidic mixing element according to any of aspects 60-63, wherein at least one mixing chamber of the mixing element comprises a reagent for mixing with the fluid sample, preferably wherein each mixing chamber comprises a reagent for mixing with the fluid sample.
64. The microfluidic mixing element according to any of aspects 60-63, wherein a width of the mixing chamber in the direction of fluid flow is at least four times a width of the narrow channel in the direction of fluid flow.
65. The microfluidic mixing element according to any of aspects 60-63, wherein the mixing chambers comprise at least one curved wall, preferably wherein the mixing chambers are round wells.
66. A nucleic acid amplification device comprising the microfluidic mixing element of any of aspects 60-65.
67. The nucleic acid amplification device of any of aspects 1-41, further comprising the microfluidic mixing element of any of aspects 60-65, preferably wherein the one or more first microfluidic conduit comprises the microfluidic mixing element.
68. The nucleic acid amplification system of any of aspects 42-49, wherein the amplification device further comprises the microfluidic mixing element of any of aspects 60-65, preferably wherein the one or more first microfluidic conduit comprises the microfluidic mixing element.

What is claimed is:

1. A nucleic acid amplification device for testing a bodily fluid sample for illness, the device comprising a housing comprising:
an inlet port for receiving a sample derived from a bodily fluid;
an incubation and detection section configured to enable the amplification and the detection of nucleic acids, the section comprising a plurality of incubation chambers;
a plurality of first microfluidic conduits connecting the inlet port with each of the incubation chambers;
a plurality of nucleic acid amplification compositions, each including a primer configured to amplify nucleic acids indicative of an illness, each of the nucleic acid amplification compositions being located along each of the first microfluidic conduits and/or in each incubation chamber, wherein the nucleic acid amplification compositions are provided in the form of a lyophilized pellet, sphere or powder; and
a heating arrangement comprising a heating element adjacent the at least one incubation chamber.

2. The amplification device according to claim 1, wherein the incubation and detection section further comprises a plurality of detection chambers configured to enable the detection of amplified nucleic acids, and the incubation and detection section further comprises a plurality of second microfluidic conduits connecting each of the incubation chambers with a respective detection chamber.

3. The amplification device according to claim 1 or 2,
wherein the device comprises at least 2, at least 3 or at least 5 incubation chambers; and/or
wherein the device comprises between 2 and 12 incubation chambers, preferably between 2 and 7, more preferably between 3 and 7, most preferably 5 incubation chambers.

4. The amplification device according to any of the previous claims,
wherein each nucleic acid amplification composition comprises a different primer configured to amplify nucleic acids indicative of a different illness; and/or
wherein the primer of a first nucleic acid amplification composition is configured to amplify nucleic acids of at least one of Chlamydia trachomatis, Neisseria gonorrhoeae, Mycoplasma genitalium or Trichomonas vaginalis, the primer of a second nucleic acid amplification composition is configured to amplify nucleic acids of a another one of Chlamydia trachomatis, Neisseria gonorrhoeae, Mycoplasma genitalium or Trichomonas vaginalis, and the primer of a third nucleic acid amplification composition is configured to amplify nucleic acids of a further one of Chlamydia trachomatis, Neisseria gonorrhoeae, Mycoplasma genitalium or Trichomonas vaginalis.

5. The amplification device according to any of the previous claims, wherein the incubation and detection section comprises at least one test strip, wherein the test strip has been functionalized to change in color upon presence of a threshold amount of DNA and/or RNA.

6. The amplification device according to any of the previous claims, wherein the incubation and detection section comprises a leuco dye, preferably a leuco dye configured to turn to a blue color to enable detection, and/or wherein the incubation and detection section comprises a rhodamine B—Cu dye which changes color in the presence of pyrophosphate.

7. A single-use nucleic acid amplification device for testing a bodily fluid sample for illness, the device comprising a housing comprising:
an inlet port for receiving a sample derived from a bodily fluid;
an incubation and detection section configured to enable the amplification and the detection of nucleic acids, the section comprising one or more incubation chambers;
one or more first microfluidic conduits connecting the inlet port with each of the one or more incubation chambers;
at least one nucleic acid amplification composition including a primer configured to amplify nucleic acids indicative of an illness, each of the nucleic acid amplification compositions being located along one of the first microfluidic conduits and/or in one of the incubation chambers; and
a heating arrangement comprising a heating element adjacent each of the one or more incubation chambers, wherein the heating element is mechanically attached and/or bonded to the housing.

8. The amplification device according to any of the previous claims,
wherein the heating element is an electrical heating element, preferably wherein the heating element is a resistive-type heating element and/or wherein the heating element is formed on a printed circuit board (PCB); and/or
wherein the heating element is a chemical heating element, the heating element being configured to produce a chemical reaction and to heat the at least one incubation chamber to a target temperature by said chemical reaction.

9. The amplification device according to any of the previous claims, wherein the amplification device further comprises
at least one plug, for example a USB plug, for connecting to an electric power supply, wherein the plug is provided in the housing; and/or
at least one cable for connecting to an electric power supply, wherein the cable is plugged into or firmly attached to the housing; and/or
at least one battery for providing energy to the heating element, wherein the battery is received in the housing and/or attached to the housing.

10. The amplification device according to any of the previous claims, wherein an electrical circuit between a power supply of the heating arrangement and the heating element is open in the absence of sample within the amplification device, and wherein the electrical circuit is closed upon the presence of sample within the amplification device, preferably wherein the sample acts as an electrical conductor to close the electrical connection between the power supply and the heating element.

11. The amplification device according to any of the previous claims, wherein the device comprises a plurality of incubation chambers, wherein the heating arrangement is configured to heat each of the incubation chambers to a respective target temperature, wherein the target temperature differs between the incubation chambers, preferably wherein one or more incubation chambers that are to be heated to a higher target temperature are arranged between incubation chambers that are to be heated to a lower target temperature.

12. The amplification device according to any of the previous claims,
wherein the heating arrangement is configured to provide a maximum temperature of 75° C., preferably a maximum temperature of 70° C., more preferably a maximum temperature of 65° C.; and/or
wherein the heating arrangement is configured to provide a minimum temperature of 50° C., preferably 55° C., more preferably at least 60° C.

13. A nucleic acid amplification system comprising:
the amplification device of any of claims 1 to 12; and
an external electronic device with a camera, preferably a handheld device such as a mobile phone or a tablet, the external device being configured to capture an image of the incubation and detection section and evaluate whether amplification of nucleic acids has been detected.

14. The amplification system according to claim 13, wherein the external device is programmed to detect a color change of a test compound, preferably wherein the external device is programmed to detect a blue color, in particular a blue color of a leuco dye; and/or the external device is programmed to detect a pink color, in particular a pink color of a rhodamine B—Cu dye.

15. The amplification system according to claim 13 or 14, wherein the housing of the amplification device further comprises at least one identification marker, preferably a QR code, wherein the external device is programmed to detect the at least one identification marker and interpret the orientation of the housing based thereon.
